(19) United States
(12) Patent Application Publication
(10) Pub. No.: US 2008/0078390 A1
(43) Pub. Date: Apr. 3, 2008

(54) Providing predetermined groups of trending parameters for display in a breathing assistance system

(75) Inventors: Gary Scott Milne, Louisville, CO (US); David Ray Hyde, Livermore, CA (US)

Correspondence Address: Covidien;IP Counsel, Respiratory & Monitoring Solutions, 60 Middleton Avenue, North Haven, CT, 06473, US

(73) Assignee: Nellcor Puritan Bennett Incorporated

(21) Appl. No.: 11/537,246

(22) Filed: Sep. 29, 2006

(51) Int. Cl. A61M 16/00

(52) U.S. Cl. 128/204.23; 128/204.21

(57) ABSTRACT

The disclosure provides a breathing assistance system that may include: a storage device operable to store multiple trending parameter groups; a user interface operable to exhibit the identifiers for at least a portion of the trending parameter groups and to receive a user selection of a particular one of the exhibited identifiers; and a display operable to display each of the trending parameters in the selected trending parameter group.

### TECHNICAL FIELD

The present disclosure relates generally to the field of medical devices, e.g., providing predetermined groups of trending parameters for display in a breathing assistance system

### BACKGROUND

Conventional breathing assistance systems typically include a gas delivery system, a patient interface to deliver gas to one or more breathing passages of the patient, and a connection system between the gas delivery system and the patient interface. Such breathing assistance systems may be used, e.g., for mechanical ventilation of a patient's lungs and/or treatment of an apnea or other medical condition.

Clinical treatment of a ventilated patient often requires that the breathing characteristics of the patient be monitored to detect the effects of a particular ventilation strategy on a patient or changes in the patient's breathing patterns. Many modern ventilators include a display that provides a visual display of various parameters regarding the patient's breathing patterns and/or the operation of the ventilator, and may allow the caregiver to adjust ventilator settings to select or adjust the ventilation strategy being implemented. For example, a ventilator may display one or more of the following parameters: airway pressure, exhaled volume, ventilation mode, type of breath, mean airway pressure, peak airway pressure, PEEP/CPAP pressure, plateau pressure, respiratory rate, I:E ratio, tidal volume, minute volume, and spontaneous minute volume.

### SUMMARY

In accordance with one embodiment of the present disclosure, a breathing assistance system may include a storage device, a user interface, and a display. The storage device may be operable to store multiple trending parameter groups, each trending parameter group including a predetermined grouping of trending parameters and having a corresponding identifier. The user interface may be operable to exhibit the identifiers for at least a portion of the trending parameter groups and to receive a user selection of a particular one of the exhibited identifiers. The display may be operable to display each of the trending parameters in the selected trending parameter group, including displaying each trending parameter versus time to indicate a trend for that trending parameter.

In accordance with another embodiment of the present disclosure, a breathing assistance system may include a gas delivery apparatus, a storage device, a user interface, and a display. The gas delivery apparatus may be configured to deliver gas toward a patient. The storage device may be operable to store multiple trending parameter groups, each trending parameter group including a predetermined grouping of trending parameters and having a corresponding identifier, and wherein the value of at least one trending parameter is a measure of a parameter of the gas delivered by the gas delivery apparatus. The user interface may be operable to exhibit the identifiers for at least a portion of the trending parameter groups and to receive a user selection of a particular one of the exhibited identifiers. The display may be operable to display each of the trending parameters in the selected trending parameter group, including displaying each trending parameter versus time to indicate a trend for that trending parameter.

In accordance with another embodiment of the present disclosure, a method for displaying trending parameters related to the operation of a breathing assistance system is provided. A plurality of identifiers may be exhibited, each identifier corresponding to one of a plurality of trending parameter groups, each trending parameter group including a predetermined grouping of trending parameters, and each trending parameter having a value that may change over time. A user selection of a particular one of the exhibited identifiers corresponding with a particular trending parameter group may be received. Each of the trending parameters in the selected trending parameter group may be displayed in response to the user selection, including displaying the value of that trending parameter versus time to indicate a trend for that trending parameter.

In accordance with another embodiment of the present disclosure, a user may operate a breathing assistance system that exhibits a plurality of identifiers, each identifier corresponding to one of a plurality of trending parameter groups, each trending parameter group including a predetermined grouping of trending parameters, and each trending parameter having a value that may change over time. The user may operating a user interface of the breathing assistance system to select one of the exhibited identifiers corresponding with a particular trending parameter group such that the trending parameters in the selected trending parameter group are displayed via a display device. The value of each trending parameter may be displayed versus time to indicate a trend for that trending parameter.

In accordance with another embodiment of the present disclosure, a computer-readable storage medium may store a set of instructions executable on a processor. The set of instructions may include instructions for exhibiting a plurality of identifiers, each identifier corresponding to one of a plurality of trending parameter groups, each trending parameter group including a predetermined grouping of trending parameters. Each trending parameter may have a value that may change over time. The set of instructions may further include instructions for receiving a user selection of a particular one of the exhibited identifiers corresponding with a particular trending parameter group. The set of instructions may further include instructions for displaying each of the trending parameters in the selected trending parameter group, including displaying the value of that trending parameter versus time to indicate a trend for that trending parameter.

In accordance with another embodiment of the present disclosure, a breathing assistance system may include: means for storing multiple trending parameter groups, means for exhibiting identifiers for at least a portion of the trending parameter groups, means for receiving a user selection of a particular one of the exhibited identifiers corresponding with a particular trending parameter group, and means for displaying each of the trending parameters in the selected trending parameter group. Each trending parameter group may include a predetermined grouping of trending parameters and have a corresponding identifier. Each trending parameter may have a value that may change over time. Displaying each trending parameter may include displaying the value of that trending parameter versus time to indicate a trend for that trending parameter.

### BRIEF DESCRIPTION OF THE DRAWINGS

Some embodiments of the disclosure may be understood by referring, in part, to the following description and the accompanying drawings wherein:

FIG. 1 illustrates an example breathing assistance system, according to one embodiment of the present disclosure;

FIG. 2 illustrates an example display device displaying a group of trending parameters, according to one example embodiment of the present disclosure;

FIG. 3 illustrates an example display including a button for opening a menu of predetermined trending parameter groups, according to one embodiment of the present disclosure;

FIG. 4 illustrates an example display indicating a selection of a particular trending parameter group for display, according to one embodiment of the present disclosure;

FIG. 5 illustrates an example display including trending data for each trending parameter in a trending parameter group selected by a user; according to one embodiment of the present disclosure; and

FIG. 6 is a flowchart illustrating a method of using a breathing assistance system offering preselected groups of trending parameters for display, according to one embodiment of the present disclosure.

### DETAILED DESCRIPTION OF THE DRAWINGS

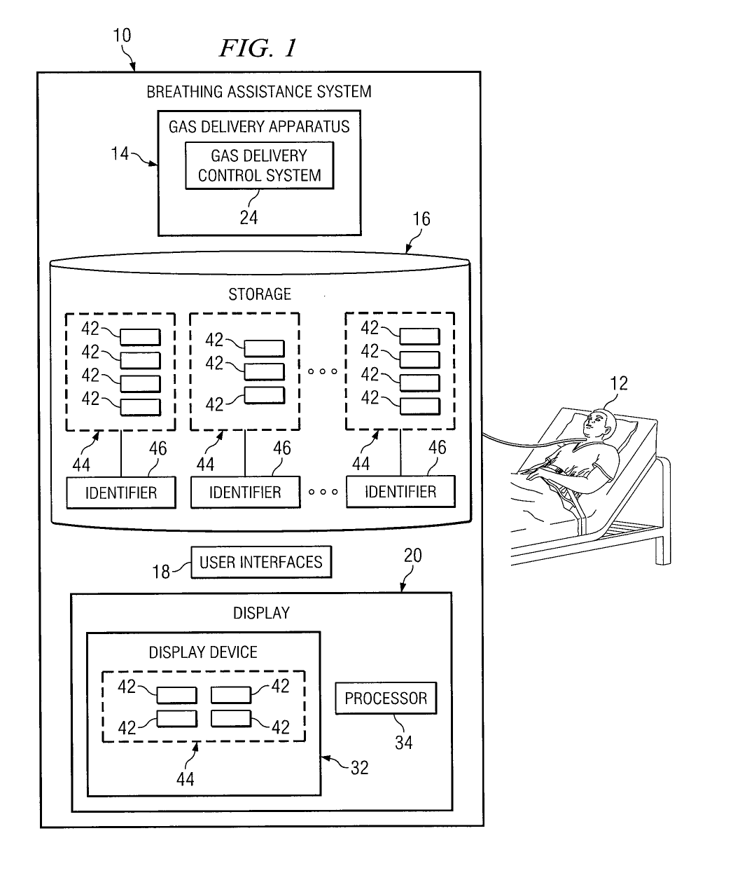

Selected embodiments of the disclosure may be understood by reference, in part, to FIGS. 1-6, wherein like numbers refer to same and like parts. The present disclosure is broadly concerned with breathing assistance systems (e.g., ventilators, CPAP systems, or BiPAP systems) adapted to provide breathing assistance to a patient (e.g., providing ventilation and/or treating an apnea or other breathing condition). The breathing assistance system may have an associated display for displaying preset groups of trending parameters. In some embodiments, a breathing assistance system may include a gas delivery apparatus (e.g., a ventilator, CPAP device, or BiPAP device) having a user interface and a display device (e.g., a GUI, touch screen, or other display). The user interface may allow a user to select from multiple predetermined groups of trending parameters a particular group of trending parameters to display. Each predetermined group of trending parameters may correspond to a particular type of treatment (e.g., a particular ventilation mode), a condition or characteristic of the patient, or any other relevant issue. Trending parameters may include parameters having values that may vary over time (e.g., ventilation frequency, patient heart rate, or exhalation pressure). When the user selects the particular group of trending parameters to display, the display device may display values for each of the trending parameters over time such that trends for each parameter may be monitored by a user, such as a caregiver.

FIG. 1 illustrates an example breathing assistance system 10 for providing breathing assistance to a patient 12, according to one embodiment of the disclosure. Breathing assistance system 10 may be generally configured to provide breathing assistance to a patient 12 (e.g., to provide ventilation and/or treat an apnea, snoring, or other breathing condition). Breathing assistance system 10 may include a gas delivery apparatus 14, a storage device 16 for storing predetermined groups of trending parameters, one or more user interfaces 18 for receiving user input, including selections of trending parameter groups to display, and a display 20 for displaying groups of trending parameters and/or other data related to breathing assistance system 10.

Gas delivery apparatus 14 may include any device or devices configured to generate, supply, and/or deliver gas (e.g., pressurized air) toward patient 12 via a connection system (e.g., a breathing circuit) and or a patient interface (e.g., a tracheal tube or mask). For example, gas delivery apparatus 14 may comprise a device capable of generating pressurized air (e.g., a ventilator, CPAP system, or BiPAP system), a wall outlet through which pressurized air may be supplied (e.g., in a hospital or clinic), one or more tanks of compressed gas, a compressor, or any other suitable source of pressurized or non-pressurized gas.

As used herein, the term “gas” may refer to any one or more gases and/or vaporized substances suitable to be delivered to and/or from a patient via one or more breathing orifices (e.g., the nose and/or mouth), such as air, nitrogen, oxygen, any other component of air, CO₂, vaporized water, vaporized medicines, and/or any combination of two or more of the above, for example. As used herein, the term “patient” may refer to any person or animal that may receive breathing assistance from system 10, regardless of the medical status, official patient status, physical location, or any other characteristic of the person. Thus, for example, patients may include persons under official medical care (e.g., hospital patients), persons not under official medical care, persons receiving care at a medical care facility, persons receiving home care, etc.

Gas delivery apparatus 14 may include a gas delivery control system 24 operable to control the breathing assistance provided by gas delivery apparatus 14 based on various input. For example, gas delivery control system 24 may regulate the pressure and/or flow of gas delivered to patient 12 based on various input received by gas delivery control system 24. Such input may include input received from an operator (e.g., via a touch screen and/or other user interfaces provided by gas delivery apparatus 14) and/or data received from one or more sensors or other electronic devices. Gas delivery control system 24 may include, or have access to, one or more processors, memory devices, and any other suitable hardware or software. The one or more memory devices may store instructions (e.g., any suitable software, algorithms, or other logic or instructions that may be executed by one or more processors) for controlling the operation of gas delivery apparatus 14, e.g., controlling ventilation support provided by gas delivery apparatus 14. Gas delivery apparatus 14 may further include any other components suitable for providing functionality related to providing breathing assistance to a patient 12. For example, gas delivery apparatus 14 may include one or more sensors, a humidifier, a nebulizer, an alarm system, and/or any other suitable components.

Display 20 may include a display device 32, a processor 34, and/or any other suitable components. Display devices 32 may include any system or device for displaying various information regarding breathing assistance system 10 (e.g., data regarding patient 12, the operation of gas delivery apparatus 14, menus, icons, selection tools and/or any other relevant data). Display device 32 may comprise any type of screen or other visual display (e.g., a touch screen display, and/or oscilloscope). Display 20 may be partially or fully integrated with, or may be physically separate from, gas delivery apparatus 14. For example, display 20 may comprise an integrated screen of a ventilator, CPAP, or BiPAP device, or a separate device such as a stand-alone monitoring device or a laptop computer.

As discussed above, display device 32 may display various parameters related to breathing assistance system 10 (e.g., data regarding patient 12, the operation of gas delivery apparatus 14, menus, icons, selection tools, and/or any other relevant data). For example, display device 32 may display any one or more of the following parameters: airway pressure, exhaled volume, ventilation mode, type of breath, mean airway pressure, peak airway pressure, PEEP/CPAP pressure, plateau pressure, respiratory rate, I:E ratio, tidal volume, minute volume, and spontaneous minute volume.

Display 20 may display various trending parameters 42. A trending parameter 42 may be any parameter that has a value that may change over time. For example, a trending parameter 42 may have a value that increases and/or decreases over time. Some trending parameters 42 may remain constant or substantially constant during particular periods of time or based on particular settings. For example, a particular trending parameter 42 may remain constant in a particular ventilation mode, but may vary over time in other ventilation modes.

In some embodiments, display 20 may simultaneously display predetermined groupings of trending parameters 42, which may be referred to as trending parameter groups 44. A trending parameter group 44 may include any group of trending parameters 42 predetermined for simultaneous display to aid a caregiver or for some other medical purpose. For example, a trending parameter group 44 may correspond to a particular mode of operation of breathing system 10 (e.g., a particular ventilation mode), a particular medical condition of patient 12, and/or any other relevant grouping of trending parameters 42.

For example, a trending parameter group 44 may be predetermined for monitoring a patient diagnosed with Adult Respiratory Distress Syndrome (ARDS). The trending parameter group 44 may include trending parameters 42 focused on lung impedance and pressure and volume relationships (e.g., set PEEP, set tidal volume, compliance, resistance, and/or plateau pressure).

As another example, a trending parameter group 44 may be predetermined for monitoring a patient being weaned from a ventilator. The trending parameter group 44 may include trending parameters 42 focused on pressure and breath timing variables and/or auto-PEEP indicators (e.g., frequency, pressure support, volume support, rapid-shallow breathing index, compliance, and/or resistance).

As another example, a trending parameter group 44 may be predetermined for monitoring a ventilator operating in a hi-level mode. The trending parameter group 44 may include trending parameters 42 focused on spontaneous breathing indicators and patient lung impedance (e.g., PEEP_{HIGH}, PEEP_{LOW}, end expiratory flow, compliance, and/or time_{LOW}).

As another example, a trending parameter group 44 may be predetermined for a particular ventilation mode: volume control plus (VC+). The trending parameter group 44 may include trending parameters 42 focused on pressure and volume relationships, breath timing and lung impedance (e.g., peak inspiratory pressure, set tidal volume, inspiratory time, compliance, and/or resistance).

As another example, a trending parameter group 44 may be predetermined for another ventilation mode: proportional assist ventilation plus (PAV+). The trending parameter group 44 may include trending parameters 42 focused on breathing and lung impedance (e.g., patient work of breathing, total work of breathing, peak inspiratory pressure, percent support, compliance, resistance, and/or intrinsic PEEP).

Trending parameter groups 44 may be predetermined or pre-selected in any suitable manner and based on any suitable data or input. For example, trending parameter groups 44 may be preprogrammed into breathing assistance system 10 based on the experience of caregivers regarding parameters suitable for monitoring in particular ventilation modes or for patients with particular medical conditions. As another example, in some embodiments, one or more trending parameter groups 44 may be configured and/or modified by a user (e.g., a technician or caregiver) as desired by the user. In such embodiments, a user interface may by provided to allow user to configure/modify trending parameter groups 44, e.g. via a series of menus, prompts, and/or other user input systems.

Trending parameter groups 44 may have corresponding identifiers 46. Each identifier 46 may correspond to a particular trending parameter group 44 and may be used by system 10 and/or a user to identify that trending parameter group 44. Identifiers 46 may include any graphic representation, icon, text, auditory signal, visible light pattern or color, and/or any other suitable identification for a trending parameter group 44. For example, the identifier 46 for a trending parameter group 44 predetermined for ventilation mode volume control plus (VC+) may consist of the text “volume control plus,” or “VC.”

Storage device 16 may include any system or device for storing trending parameter groups 44 and/or other data. In some embodiments, storage device 16 may include electrically-programmable read-only memory (EPROM), a field-programmable gate array (FPGA), a computer disk, a flash drive, punch cards or any other system suitable for such storage. Storage device 16 may store trending parameters 42, trending parameter groups 44, identifiers 46, and/or correlations between identifiers 46 and trending parameter groups 44. Storage device 16 may be coupled to, integrated with, or otherwise associated with gas delivery apparatus 14, display 20, or any other component of breathing assistance system 10.

Processor 34 may include any system or device for executing code or logic instructions (e.g., software or firmware) for controlling display device 32, such as a microcontroller, a digital signal processor (DSP), an application specific integrated controller (ASIC), electrically-programmable read-only memory (EPROM), or a field-programmable gate array (FPGA), for example. Processor 34 may be the same processor used for gas delivery control system 24, or may be a separate processor.

User interfaces 18 may include any systems or devices allowing a user to input data or otherwise interact with system 10, including allowing the user to select particular trending parameter groups 44 to display. For example, one or more user interfaces 18 may be physical interfaces (e.g., physical buttons, knobs, sliders, dials, levers, or switches) provided by gas delivery apparatus 14. As another example, one or more user interfaces 18 may be provided by a graphical user interface (GUI), such as a touch screen display (e.g., on display device 32). User interfaces 18 may be coupled to, integrated with, or otherwise associated with display 20, gas delivery control system 24, and/or gas delivery apparatus 14.

In some embodiments, user interfaces 18 may be operable to exhibit one or more identifiers 46 corresponding with one or more trending parameter groups 44. For example, in the case of physical user interfaces 18 (e.g., physical buttons, knobs, sliders, dials, levers, or switches), various identifiers 46 may be written or otherwise displayed on the physical user interfaces. As another example, in the case of a touch screen display, various identifiers 46 may be displayed on the screen.

In some embodiments, user interfaces 18 may allow a user to select a particular identifier 46 corresponding with a particular trending parameter group 44 that the user wishes to have displayed via display device 32. For example, a user may make a selection by touching a touch screen, by using physical controls, or some combination thereof.

As discussed above, display 20 may display various information regarding breathing assistance system 10 (e.g., data regarding patient 12, the operation of gas delivery apparatus 14, menus, icons, selection tools and/or any other relevant data). In particular, display 20 may display the trending parameters 42 included in a trending parameter group 44 selected by a user for display. Display 20 may indicate a trend for particular parameters 42 by displaying a plot of each parameter 42 versus time. Display 20 may indicate a trend of other parameters 42 in any other suitable manner that indicates to a user a trend of such a parameter 42 over time, e.g., using any suitable graphics, text, numerical values, colors, or other indications.

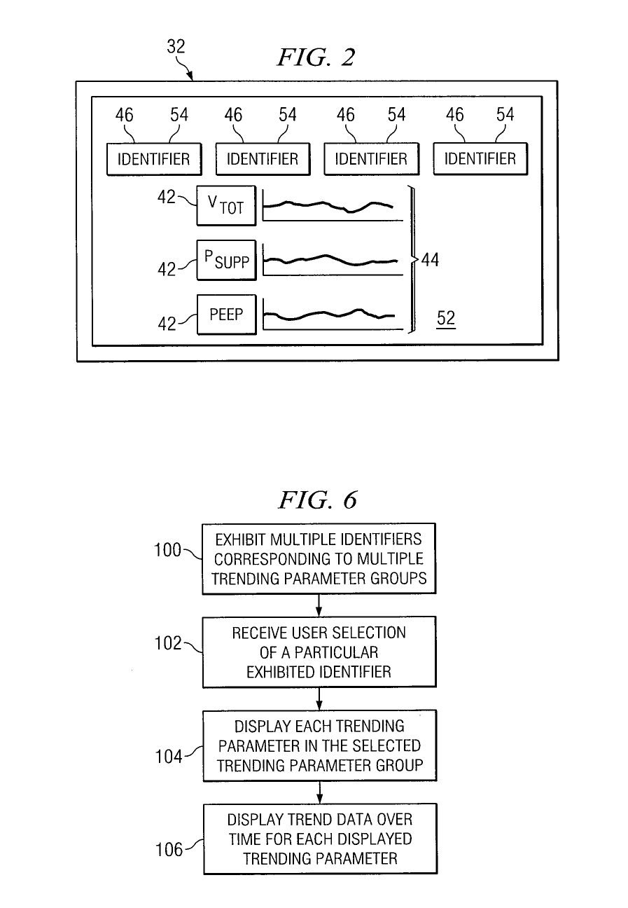

FIG. 2 illustrates an example display device 32 displaying a particular trending parameter group 44, according to one example embodiment of the present disclosure. In the illustrated embodiment, display device 32 includes a touch screen GUI display 52 which displays a number of selectable buttons 54, which operate as user interfaces 18, e.g., for navigating through screens or displays and/or for selecting, configuring, and/or modifying various parameters regarding system 10. According to another embodiment of the present disclosure, display 20 may include one or more physical user interfaces 18 (e.g., physical buttons, knobs, and/or switches), electrical interfaces (e.g., a desktop computer, laptop computer, or network terminal), and/or other means of interface (e.g., a wireless control pad or a PDA).

In the illustrated embodiment, a user may use buttons 54 to display and/or select identifiers 46 corresponding to various trending parameter groups 44. For example, as shown in FIG. 2, display 52 may display a set buttons 54 that each display a particular identifier 46. As another example, one or more buttons 54 may be used to open a window or menu (e.g., a drop-down menu) displaying a list of identifiers 46. When the user selects a particular displayed identifier 46 (using buttons 54 or other user interface 18), the trending parameters 42 included in the trending parameter group 44 corresponding to the selected identifier may be displayed (e.g., simultaneously or otherwise) in display 52. As discussed above, a value for each trending parameter 42 may be displayed in any suitable manner to indicate a trend for that parameter 42 over time. For example, in the illustrated embodiment, a plot of each trending parameter 42 over time is displayed in display 52.

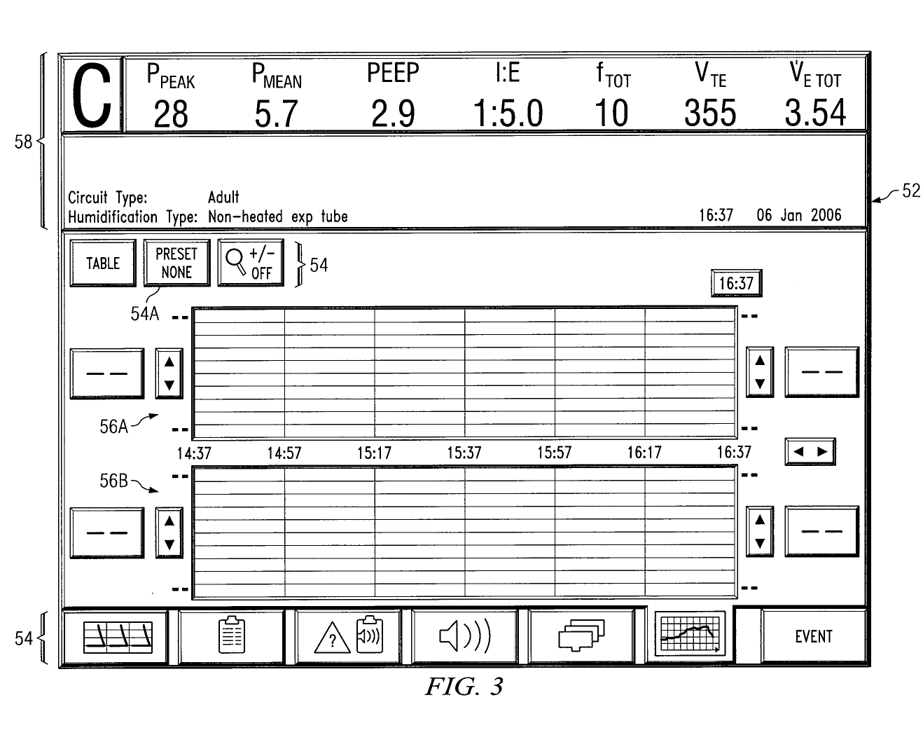

FIG. 3 illustrates an example GUI display 52 including a button for opening a menu of predetermined trending parameter groups 44, according to one embodiment of the present disclosure. In this embodiment, GUI display 52 may include a number of buttons 54 operable to receive user selections, charting areas 56 (here, 56A and 56B) for displaying trending data for one or more trending parameters 42, and various other data 58, e.g., data indicating various parameter settings or measured values. GUI display 52 may include number and/or type charting areas 56 for displaying trending data for trending parameters 42 in any suitable manner.

A menu button 54A may be used for selecting a particular preset trending parameter group 44 to display, and may indicate the currently selected trending parameter group 44. As shown in FIG. 3, no preset trending parameter group 44 has been selected (as indicated by “NONE”), and thus no trending parameters 42 or trending data are displayed in charting areas 56A or 56B.

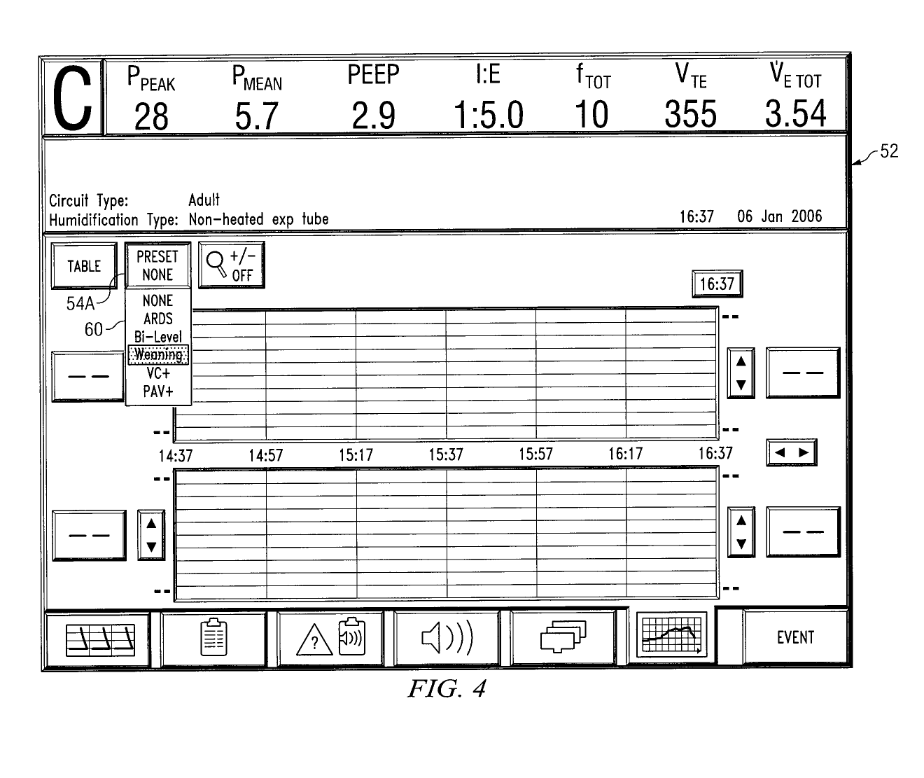

As shown in FIG. 4, menu button 54A may be selected (e.g., pressed) by a user (e.g., by touching button 54 in a touch screen configuration, by using physical controls to select menu button 54A, or by a combination of touch screen and physical control interaction) to open a drop-down menu 60. Drop down menu 60 includes a number of identifiers 46 each corresponding to a preset trending parameter group 44 available for selection. In this example, identifiers 46 indicate the following trending parameter groups 44 available for selection: “ARDS,” “Bi-Level,” “Weaning,” “VC+,” and “PAV+.”

The user may highlight and/or select an identifier 46 in any suitable manner (e.g., using a touch screen and/or physical controls). As shown in FIG. 4, the user has highlighted the “Weaning” identifier 46 for selection. When the user selects “Weaning” identifier 46, display 52 will display the trending parameters 42 included in the preset “Weaning” trending parameter group 44, as well as trending data for such trending parameters 42 in charting areas 56.

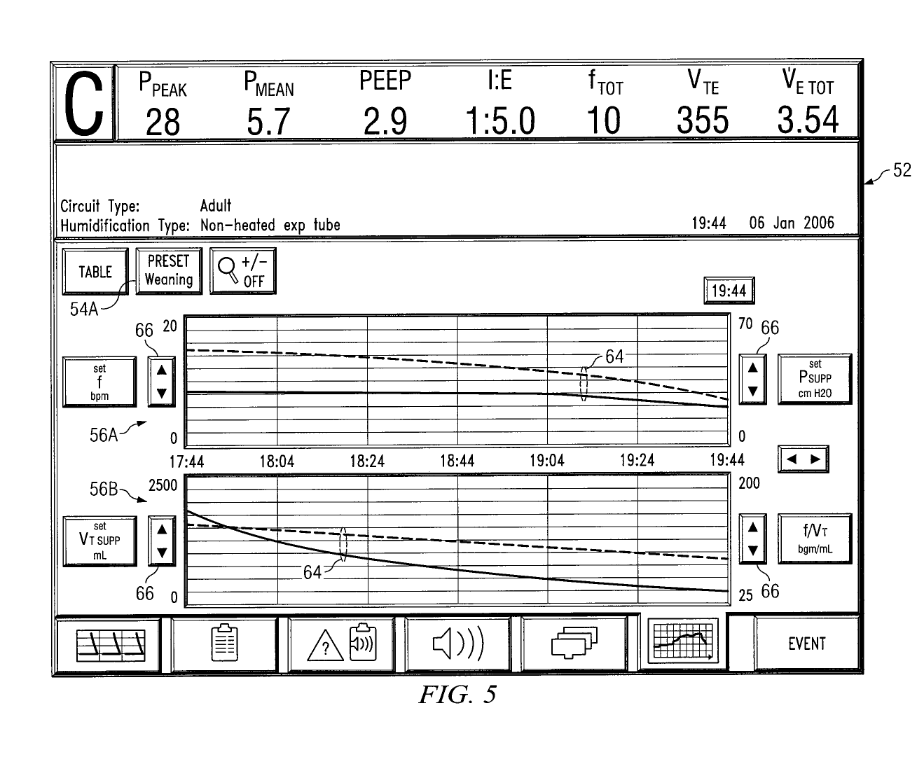

FIG. 5 illustrates display 52 after the user has selected the “Weaning” identifier 46 and some time has expired. In this example, the “Weaning” trending parameter group 44 includes four trending parameters 42: “f”, “P_{SUPP}”, “V_{T-SUPP}”, and f/V_{T},” as indicated in charting areas 56A and 56B. In this embodiment, each charting area 56A and 56B is used to display a pair trending parameters 42. Trending data 64 for each pair may be distinguished from each other by color, line type, or in any other manner. In other embodiments, trending parameter 42 may have a separate charting area 56. As shown in FIG. 5, display 52 may also include scale adjusters 66 that may be selected by a user to adjust the scale for the relevant data, e.g., to keep the trend data from moving outside of the relevant charting area (e.g., when a value becomes too low or too high).

FIG. 6 is a flowchart illustrating a method of displaying trending parameter groups 44 related to the operation of breathing assistance system 10, according to one embodiment of the present disclosure. At step 100, breathing assistance system 10 may exhibit a plurality of identifiers 46. Each identifier 46 may correspond to one of a plurality of trending parameter groups 44. Each trending parameter group 44 may include a predetermined grouping of trending parameters 42. Each trending parameter 42 may have a value that may change over time. At step 102, breathing assistance system 10 may receive a user selection of a particular one of the exhibited identifiers 46 corresponding with a particular trending parameter group 44. At step 104, breathing assistance system 10 may display each of the trending parameters 42 in the selected trending parameter group 44. As shown at step 106, displaying each trending parameter 42 may include displaying the value of that trending parameter 42 versus time to indicate a trend for that trending parameter.

It will be appreciated that while the disclosure is particularly described in the context of breathing assistance systems, the apparatuses, techniques, and methods disclosed herein may be similarly applied in other contexts. Additionally, it should be understood that various changes, substitutions and alterations can be made herein without departing from the spirit and scope of the disclosure as illustrated by the following claims.

What is claimed is:

1. A breathing assistance system, comprising:
a storage device operable to store multiple trending parameter groups, each trending parameter group including a predetermined grouping of trending parameters and having a corresponding identifier, each trending parameter having a value that may change over time;
a user interface operable to:
exhibit the identifiers for at least a portion of the trending parameter groups; and
receive a user selection of a particular one of the exhibited identifiers corresponding with a particular trending parameter group; and
a display operable to display each of the trending parameters in the selected trending parameter group, wherein displaying each trending parameter includes displaying the value of that trending parameter versus time to indicate a trend for that trending parameter.

2. A breathing assistance system according to claim 1, wherein at least one identifier corresponds to a mode of operation of the breathing assistance system.

3. A breathing assistance system according to claim 1, wherein at least one identifier corresponds to a patient condition.

4. A breathing assistance system according to claim 1, wherein at least one identifier corresponds to a ventilation mode.

5. A breathing assistance system according to claim 1, wherein the user interface includes a graphic user interface.

6. A breathing assistance system according to claim 1, wherein the user interface includes one or more manual input devices separate from the display, the one or more manual input devices configured to facilitate the user selection.

7. A breathing assistance system according to claim 6, wherein the one or more manual input devices configured to facilitate the user selection are selected from the group consisting of a dial, a knob, a button, a slider, a lever, and a switch.

8. A breathing assistance system according to claim 1, wherein multiple trending parameter groups may include at least one same trending parameter.

9. A breathing assistance system according to claim 1, wherein displaying the value of a particular trending parameter versus time to indicate a trend for that trending parameter comprises displaying a plot of the particular trending parameter versus time.

10. A breathing assistance system according to claim 1, wherein:
displaying the value of a first trending parameter versus time to indicate a trend for the first trending parameter comprises displaying a first plot of the first trending parameter versus time;
displaying the value of a second trending parameter versus time to indicate a trend for the second trending parameter comprises displaying a second plot of the second trending parameter versus time; and
the first plot and the second plot are displayed on the same graph.

11. A breathing assistance system, comprising:
a gas delivery apparatus configured to deliver gas toward a patient;
a storage device operable to store multiple trending parameter groups, each trending parameter group including a predetermined grouping of trending parameters and having a corresponding identifier, each trending parameter having a value that may change over time, and wherein the value for at least one trending parameter is a measure of a parameter of the gas delivered by the gas delivery apparatus;
a user interface operable to:
exhibit the identifiers for at least a portion of the trending parameter groups; and
receive a user selection of a particular one of the exhibited identifiers corresponding with a particular trending parameter group; and
a display operable to display each of the trending parameters in the selected trending parameter group, wherein displaying each trending parameter includes displaying the value of that trending parameter versus time to indicate a trend for that trending parameter.

12. A breathing assistance system according to claim 11, wherein the values of at least a portion of the trending parameters are based at least on data from one or more sensors.

13. A method for displaying trending parameters related to the operation of a breathing assistance system, the method comprising:
exhibiting a plurality of identifiers, each identifier corresponding to one of a plurality of trending parameter groups, each trending parameter group including a predetermined grouping of trending parameters, each trending parameter having a value that may change over time;
receiving a user selection of a particular one of the exhibited identifiers corresponding with a particular trending parameter group; and
displaying each of the trending parameters in the selected trending parameter group, wherein displaying each trending parameter includes displaying the value of that trending parameter versus time to indicate a trend for that trending parameter.

14. A method according to claim 13, wherein at least one identifier corresponds to a mode of operation of the breathing assistance system.

15. A method according to claim 13, wherein at least one identifier corresponds to a patient condition.

16. A method according to claim 13, wherein at least one identifier corresponds to a ventilation mode.

17. A method according to claim 13, wherein the user selection is received via a graphic user interface.

18. A method according to claim 13, wherein the user selection is received via a user interface including a display and one or more manual input devices separate from the display, the one or more manual input devices configured to facilitate the user selection.

19. A method according to claim 18, wherein the one or more manual input devices configured to facilitate the user selection are selected from the group consisting of a dial, a knob, a button, a slider, a lever, and a switch.

20. A method according to claim 13, wherein multiple trending parameter groups may include at least one same trending parameter.

21. A method according to claim 13, wherein displaying the value of a particular trending parameter versus time to indicate a trend for that trending parameter comprises displaying a plot of the particular trending parameter versus time.

22. A method according to claim 13, wherein:
displaying the value of a first trending parameter versus time to indicate a trend for the first trending parameter comprises displaying a first plot of the first trending parameter versus time;
displaying the value of a second trending parameter versus time to indicate a trend for the second trending parameter comprises displaying a second plot of the second trending parameter versus time; and
the first plot and the second plot are displayed on the same graph.

23. A method for operating a breathing assistance system, comprising:
operating a breathing assistance system that exhibits a plurality of identifiers, each identifier corresponding to one of a plurality of trending parameter groups, each trending parameter group including a predetermined grouping of trending parameters, each trending parameter having a value that may change over time; and
operating a user interface of the breathing assistance system to select one of the exhibited identifiers corresponding with a particular trending parameter group such that the trending parameters in the selected trending parameter group are displayed via a display device, wherein the value of each trending parameter is displayed versus time to indicate a trend for that trending parameter.

24. A computer-readable storage medium storing a set of instructions executable on a processor, the set of instructions comprising:
instructions for exhibiting a plurality of identifiers, each identifier corresponding to one of a plurality of trending parameter groups, each trending parameter group including a predetermined grouping of trending parameters, each trending parameter having a value that may change over time;
instructions for receiving a user selection of a particular one of the exhibited identifiers corresponding with a particular trending parameter group; and
instructions for displaying each of the trending parameters in the selected trending parameter group, wherein displaying each trending parameter includes displaying the value of that trending parameter versus time to indicate a trend for that trending parameter.

25. A breathing assistance system, comprising:
means for storing multiple trending parameter groups, each trending parameter group including a predetermined grouping of trending parameters and having a corresponding identifier, each trending parameter having a value that may change over time;
means for exhibiting the identifiers for at least a portion of the trending parameter groups; and
means for receiving a user selection of a particular one of the exhibited identifiers corresponding with a particular trending parameter group; and
means for displaying each of the trending parameters in the selected trending parameter group, wherein displaying each trending parameter includes displaying the value of that trending parameter versus time to indicate a trend for that trending parameter.
